(19) United States
(12) Patent Application Publication
(10) Pub. No.: US 2018/0116626 A1
(43) Pub. Date: May 3, 2018

(54) Heart Activity Detector for Early Detection of Heart Diseases

(71) Applicant: AWAIRE INC., San Jose, CA (US)

(72) Inventors: Anurag Darbari, San Jose, CA (US); Archana Darbari, San Jose, CA (US)

(21) Appl. No.: 15/802,374

(22) Filed: Nov. 2, 2017

Related U.S. Application Data
(60) Provisional application No. 62/416,707, filed on Nov. 3, 2016.

(51) Int. Cl. A61B 7/04; A61B 5/00; A61B 5/0205; A61B 7/02

(52) U.S. Cl. CPC A61B 7/04; A61B 5/7207; A61B 5/02055; A61B 5/026; A61B 5/6823; A61B 7/026; A61B 5/6898

(57) ABSTRACT

In an embodiment, a heart activity detector is provided. In the embodiment, the heart activity detector is comprised of a multi-channel stethoscope connected to one or more processors. The multi-channel stethoscope contains an sensors to monitor each of the heart valves of a patient or user, and detect heart diseases The heart activity detector is further provided with a means to connect to a mobile communication device.

### CROSS-REFERENCE TO RELATED APPLICATION(S)

The present application claims priority to U.S. Provisional Patent Application No. 62/416,707 filed on Nov. 3, 2016, entitled “Heart Activity Detector for Early Detection of Heart Diseases” the entire disclosure of which is incorporated by reference herein.

### BACKGROUND OF THE INVENTION

Since early 1800's, the acoustic stethoscope has been used as the primary tool to diagnose heart problems. A stethoscope operates by transmitting heart sound from the chest piece, via air-filled hollow tubes, to the physician's ear for interpretation and analysis. This technique is subjective, as there is no standard calibration technique to ensure the stethoscope used by physician works accurately. Additionally, interpretation of the sound signals heard can vary among doctors of different expertise. Finally, the acoustic stethoscope has an extremely low sound level, and ambient noise mixing with the device provides distorted sound, which may result in an inaccurate diagnosis.

In today's modern world, physicians are pressed for time. It may be difficult to spend enough time with a patient to ensure a proper diagnosis. When a patient feels discomfort or pain in his/her chest, he/she is a referred to a cardiologist. The cardiologist makes a diagnosis using specialized medical equipment, trained technicians, and blood work. However, this is a time consuming and expensive process, and sometimes irreversible damage may have already happened, in the interim, to the patient. This damage may have been avoided had the patient received regular monitoring and care at the first sign of a heart irregularity.

Currently, no consumer device exists in the market which accurately monitors heart health. Some wearable devices, such smart-watch or fitness-bands, monitor heart rate. However, this information, alone, is too little to fully give insight into the condition of a patient's heart or indicate heart disease.

Blood pressure monitoring devices with an integrated heart rate monitor and pulse oximeter can provide metrics of blood vessel wall pressure, blood volume flow, and heart rate variability. However, this information does not provide comprehensive heart health information, does little to provide information of a heart's operation or indicate possible heart disease.

Based on the foregoing, there is a need in the art for a device which captures biological signals generated by the heart. Further, what is desired is a device which can analyze biological signals using artificial intelligence/machine learning techniques and provide output classification data to the user for self-monitoring and early detection of hear irregularities and disease.

### SUMMARY OF THE INVENTION

In an embodiment of the present invention, a heart activity detector is provided. In the embodiment, the heart activity detector is comprised of one or more processors. The processors are connected to a multi-channel stethoscope.

In an embodiment the multi-channel stethoscope includes a plurality of audio sensors, electrocardiogram, and pressure wave sensors. The audio sensors include an aortic valve sensor, a pulmonary valve sensor, a tricuspid valve sensor, and a mitral valve sensor. In the embodiment the heart activity device is further provided with a means to connect to a mobile communication device.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a perspective view of the heart activity detector in use, according to an embodiment of the present invention;

FIG. 2 is a data path representation of the heart activity detector, according to an embodiment of the present invention;

FIG. 3 is an electrical block diagram of the heart activity detector, according to an embodiment of the present invention;

FIG. 4 is a block diagram of the artificial intelligence engine of the heart activity detector, according to an embodiment of the present invention;

FIG. 5 is a data path representation of the artificial intelligence engine of the heart activity detector, according to an embodiment of the present invention;

FIG. 6 is a perspective view of the heart activity detector in use, according to an embodiment of the present invention;

FIG. 7 is a data path representation of the Non-Contact Bio Potential data of the heart activity detector, according to an embodiment of the present invention;

FIG. 8 is an electrical block diagram of the quadrophonic acoustic sensor of the heart activity detector, according to an embodiment of the present invention;

FIG. 9 is a graphical representation of the heart sounds captured by the heart activity detector, according to an embodiment of the present invention;

FIG. 10 is a flowchart representing classification of the heart activity detector, as Normal or Abnormal, according to an embodiment of the present invention;

FIG. 11 is a flowchart representing data classifier of the heart activity detector, according to an embodiment of the present invention;

FIG. 12 is a perspective view of the heart activity detector, packed as a smartphone cover, according to an embodiment of the present invention;

FIG. 13 is a perspective view of the heart activity detector, packaged as a wearable device, according to an embodiment of the present invention;

FIG. 14 is a perspective view of the heart activity detector as a wearable device in use, according to an embodiment of the present invention;

FIG. 15 is a perspective view of the heart activity detector, as a standalone device, according to an embodiment of the present invention;

FIG. 16 is a flowchart representing the accelerator design of the valid heart activity detection, according to an embodiment of the present invention;

FIG. 17 is a flowchart representing execution of the heart activity detector, according to an embodiment of the present invention;

FIG. 18 is a flowchart representing the place assistance mechanism of the heart activity detector, according to an embodiment of the present invention;

FIG. 19 is a flowchart representing the place assistance mechanism of the heart activity detector, according to an embodiment of the present invention; and

FIG. 20 is a flowchart of the artificial intelligence engine of the heart activity detector, according to an embodiment of the present invention.

### DETAILED DESCRIPTION OF PREFERRED EMBODIMENTS

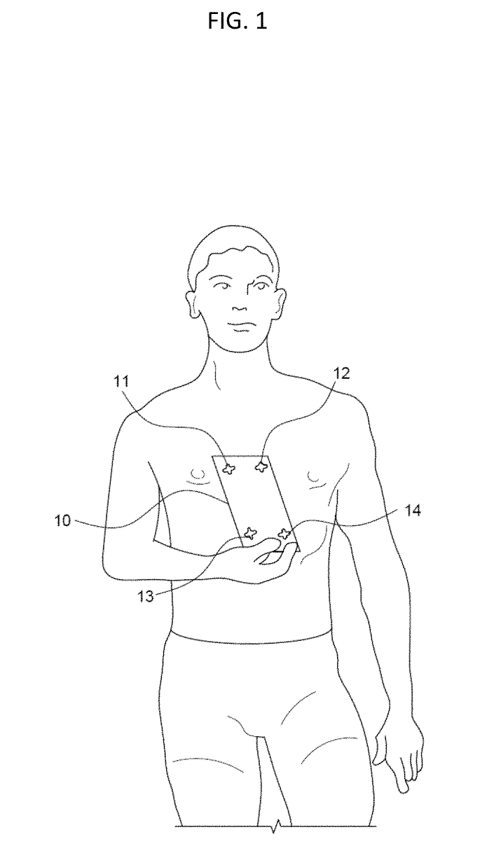

Preferred embodiments of the present invention and their advantages may be understood by referring to FIGS. 1-20, wherein like reference numerals refer to like elements.

In reference to FIG. 1, an embodiment of the present invention is shown as a smart phone or tablet configured with a multi-channel stethoscope 10. In an exemplary embodiment, the multi-channel stethoscope is provided with four audio sensors. In the embodiment, those audio sensors include an Aortic Valve sensor 11, a pulmonary valve sensor 12, a tricuspid valve sensor 13, and a mitral valve sensor 14. Each of the sensors is configured to capture sound generated by the respective heart valve, e.g. the atrial valve sensor is configured to capture sounds produced by the atrial heart valve of the user or patient. In an embodiment, the multi-channel stethoscope is further configured with mechanical event sensors, an electrocardiogram (ECG) sensor (1-wire ECG), an ambient noise capturing microphone, a gyroscope, an accelerometer, and a temperature sensor.

In an embodiment, the multi-channel stethoscope is configured to attach to an existing smart phone, tablet, or other electronic device. In another embodiment, the multi-channel stethoscope is provided as a standalone unit capable of pairing with a smart phone, tablet, computer, or other electronic device via a wired or wireless connection.

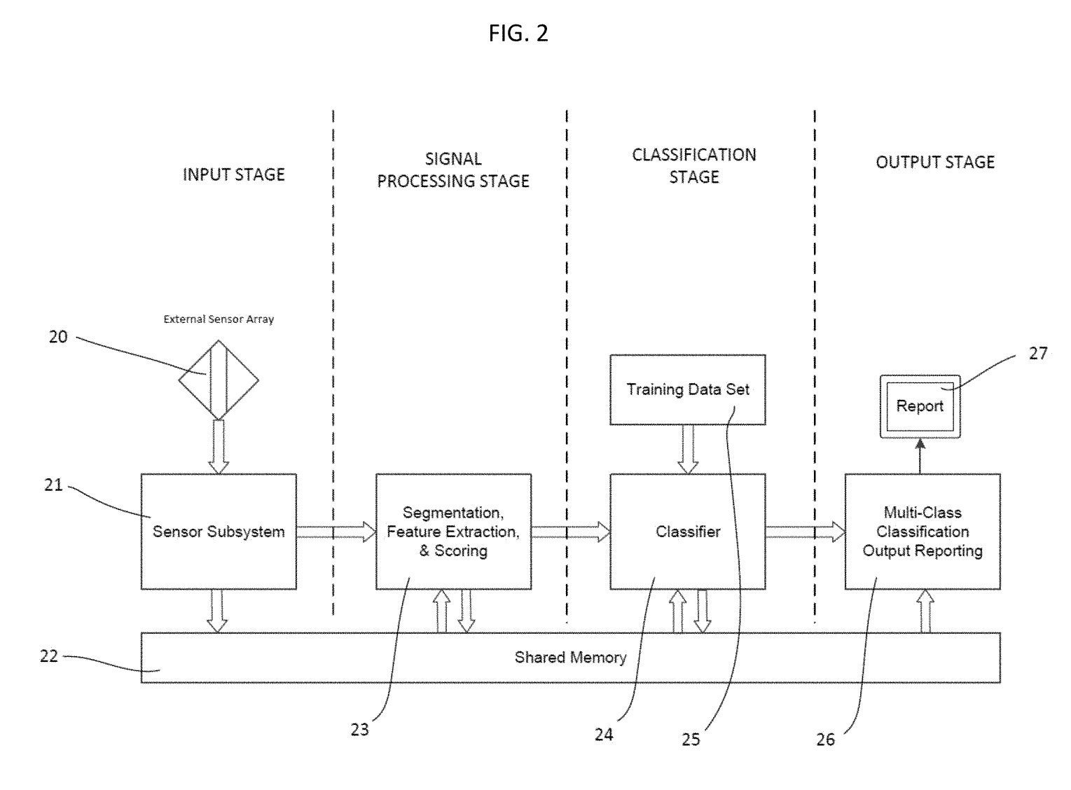

In reference to FIG. 2, an embodiment of schematic shows the data path from the sensor to classifier. In this embodiment, data is streamed from the array of sensors 20, which primarily includes heart sound, heart event detection, and electrocardiogram data, captured by sensors, to the sensor subsystem 21. The sensor subsystem 21 captures, filters, and sends the data captured from the external sensor array 20 to the shared memory 22. Acquired data is processed by signal processing block 23. Signal processing block 23 performs segmentation, decomposition, and feature extraction. The processed signal is then recorded back into the shared memory 22 and is used by the classifier 24. The classifier 24 analyzes the data to classify the acquired signals from the heart as either normal or abnormal, classify heart diseases. The classified data is then recorded to the shared memory 22 and transmitted to report generation program 26. The report generation program 26 creates a readable report 27 able to be viewed by the user, patient, or doctor. The report 27 will alert the user, patient, or doctor to abnormal heart activity, both valvular or ischemia, and predict the possibility of congestive heart failure.

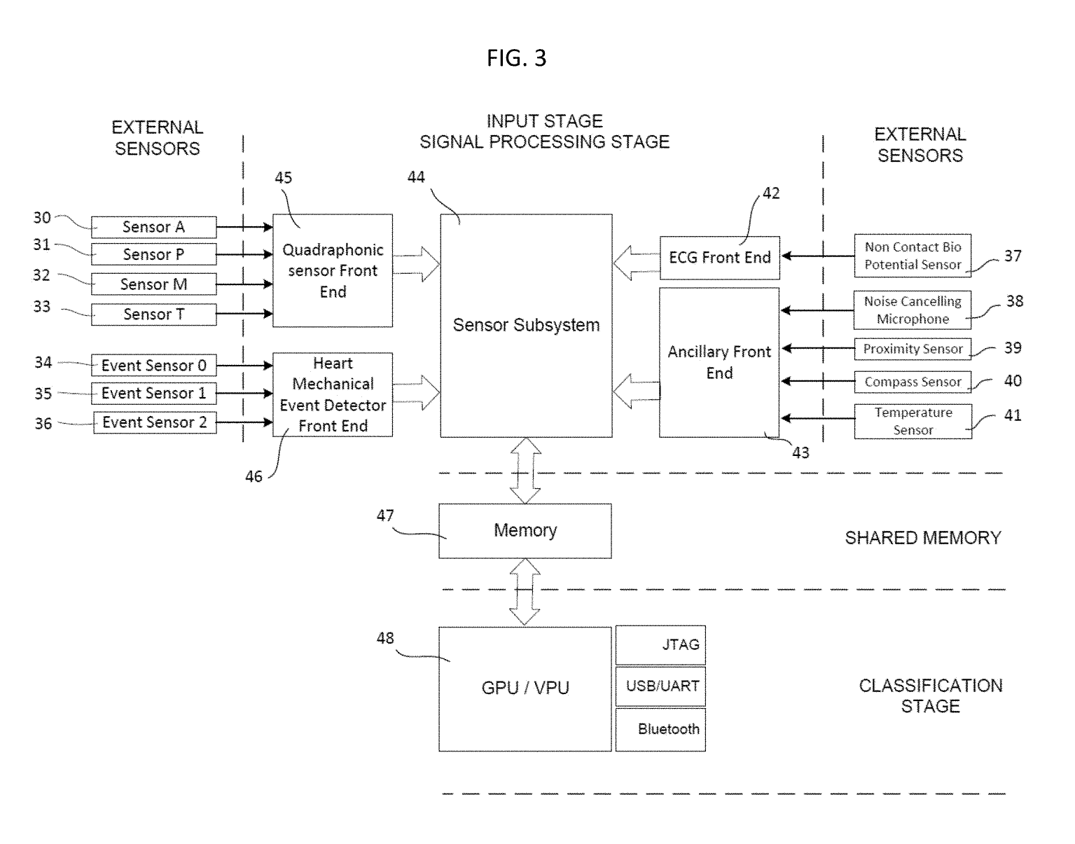

In reference to FIG. 3, a block diagram representing the electrical construction of the multi-channel stethoscope is shown, according to an embodiment of the present invention. In the embodiment, data from the phonocardiogram audio sensors positioned to receive. heart sounds generated from the interplay of the dynamic events associated with the contraction and relaxation of the atria and ventricles, as well as the valve movements, and blood flow. The heart sound data is received by the aortic sensor 30, pulmonary sensor 31, tricuspid sensor 32, and mitral sensor 33. In a specific embodiment, each sensor is capable of capturing sound between the infrasound and ultrasound region, i.e. 4 Hz to 40 Khz. In an embodiment, the phonographic audio sensors are further provided to capture respiratory sound. The sound data acquired by the phonocardiogram sensors is then transmitted to the quadraphonic front end 45, which filters, amplifies, and digitizes the sound data before transmitting the data to the sensor subsystem 44. In an embodiment in which respiratory sound is calculated, the quadraphonic front end 45 is further provided to compute respiration rates and gauge activity levels.

Again, with reference to FIG. 3, according to an embodiment, the multi-channel stethoscope is further provided with mechanical event sensors or pressure wave sensors 34, 35, and 36 which captures heart subtle movements at the surface of the chest. These sensors are placed toward the apex of the heart, to capture left and right ventricle movements. Heart Mechanical Event detector front end 46 performs signal conditioning, which includes filtering, amplification and data conversion before being transmitted to the sensor subsystem 44.

In an embodiment, the multi-channel stethoscope is further provided with a non-contact bio potential sensor 37 which captures a 1-wire electrocardiogram (ECG) signal generated by the user's heart. The ECG data provides a quick method for the detection of arrhythmia. In an embodiment, the non-contact bio potential sensor is alongside phonocardiogram audio sensors, preferably the aortic valve sensor 30 or pulmonary valve sensor 31. The ECG data is received by the ECG front end 42 where it performs signal conditioning, which includes filtering, amplification and data conversion before transmission to the sensor subsystem 44.

In an embodiment, the multi-channel stethoscope is further provided with multiple ancillary sensors. In an embodiment, the ancillary sensors include 2 noise cancelling microphones 38 provided to capture ambient noise which is filtered out from the recordation of sound data captured by the phonocardiogram audio sensors. A first microphone is provided to capture noise from the environment. A second microphone is provided to capture respiration noise, patient movements of the sensors, and acoustic dampening through the bones and tissues. The sound recorded from the noise cancelling microphones provides a baseline for which the filters can eliminate noise from the captured sound recording. Further ancillary sensors include a proximity sensor 39 to detect the placement of the multi-channel stethoscope upon the user's body, movement sensors 40 comprising of at least one gyroscope and at least one accelerometer to detect movement of the multi-channel stethoscope and user, and a temperature sensor 41 to monitor the user's body temperature. The data received by the ancillary sensors is transmitted to the ancillary front end 43 for processing before being transmitted to the sensor subsystem 44.

In an embodiment, the sensor subsystem 44 performs multi-channel data acquisition, and signal processing tasks, before transmitting the processed data to the shared memory bank 47. In an embodiment, the shared memory bank 47 then transmits the data captured to the vector processing unit (VPU) or graphic processing unit (GPU) 48 wherein the data is classified and may be transmitted to and external device or processor.

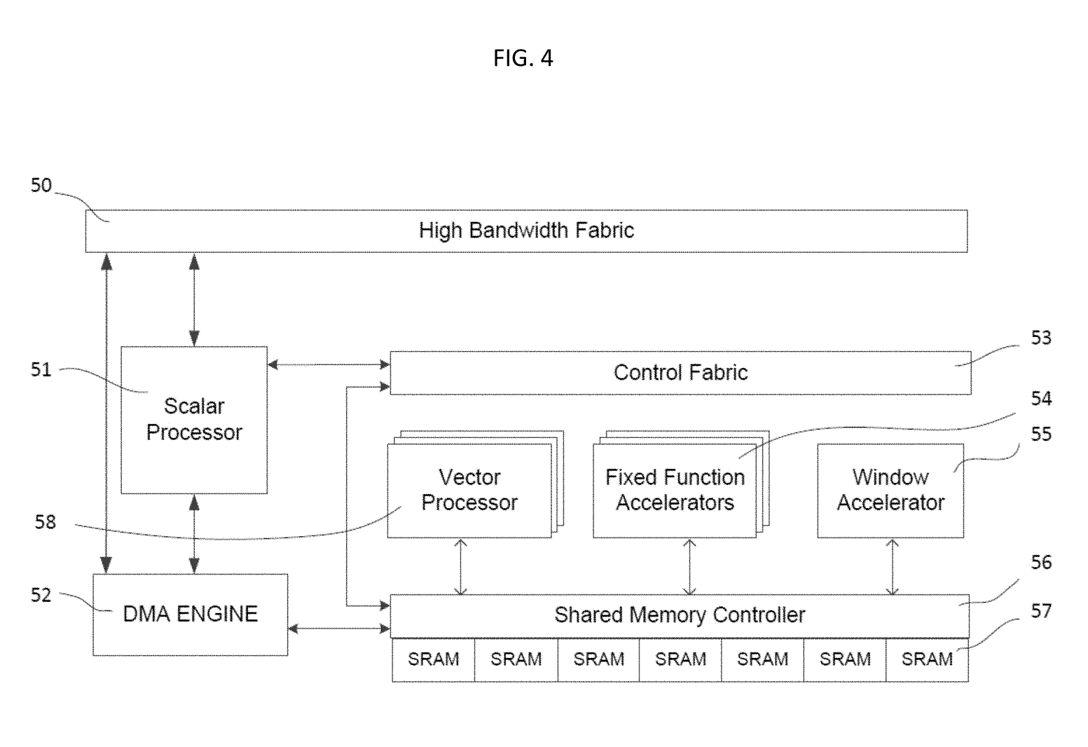

In an embodiment, in reference to FIG. 4, a block diagram is shown to represent the VPU or artificial intelligence (AI) engine and its application. In the embodiment, the high band width fabric 50 is provided to transmit signal data to the scalar processor 51 via the DMA engine 52. In an embodiment, the scalar processor 51 is a 32-bit microcontroller. The scalar processor then transmits the data to the control fabric 53 which is provided as an interface between the scalar processor 51, vector processor 58, and fixed function accelerators 54. In an embodiment, the control fabric 53 transmits data to and from the shared memory bank 56. The shared memory bank 56 stores data from the sensor rapid access memory 57 to be transmitted to the vector processor 58, fixed function accelerators 54, and window accelerator 55.

In an embodiment, the vector processor 58 is comprised of multi core arithmetic logic units (ALUs) running in parallel to perform classification tasks. Further in an embodiment, the fixed function accelerators 54 accelerate fixed task sequences, and the window accelerator 55 performs cardiac cycle detection by decoding heart sounds and electrograms

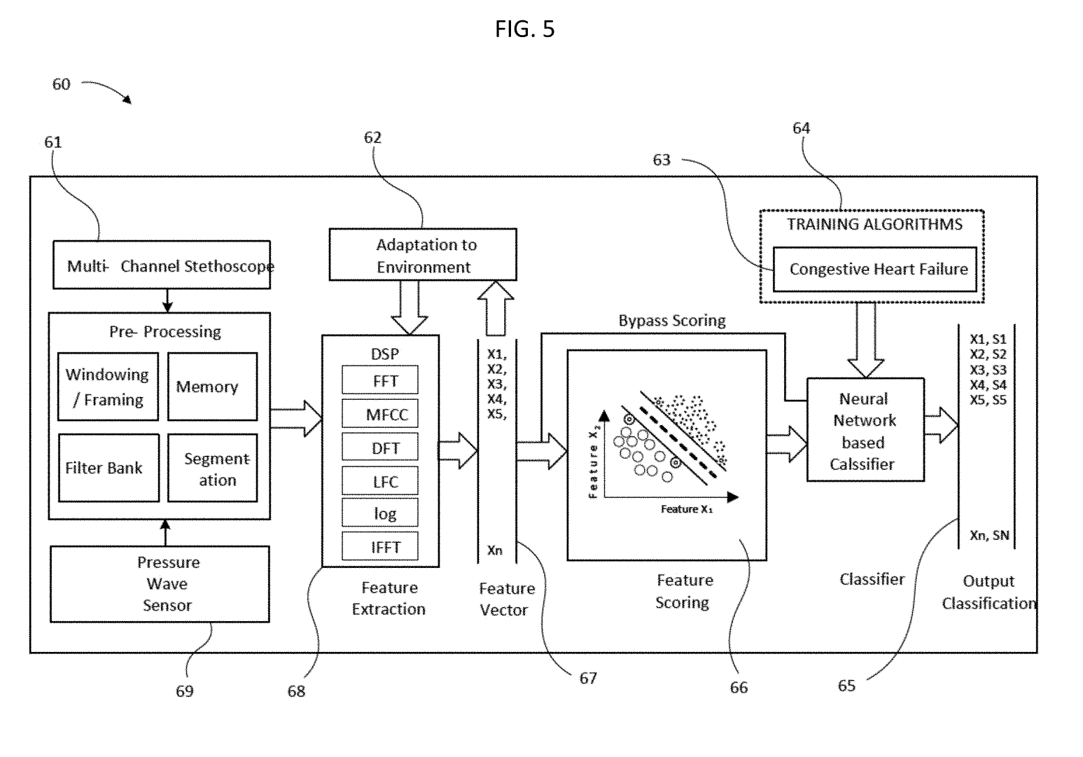

In an embodiment, with reference to FIG. 5, a data path within the AI engine 60 is represented. In the embodiment, the data received by the multi-channel stethoscope 61 and pressure wave sensor 69 is transmitted to be pre-processed, filtered, recorded, framed, and segmented before being transmitted to the digital signal processor (DSP) block 68 to be digitized and feature extracted. The DSP block 68 further receives information from adaptation block 62 which provides data signals produced by environmental and ambient noise, wherein the DSP block uses the data received from the adaptation block to compute baselines and eliminate noise signals.

Further referring to FIG. 5, in an embodiment, the DSP block 68 transmits the features extracted from the sensor data signals into a feature vector output 67. The output feature vectors are then transmitted to a feature scoring block 66 to determine a scoring value for heart diseases and other heart conditions. This score is then passed to the classifier along with feature vector outputs which bypass scoring to be compared to training algorithms 64 and example data sets 63 to be processed and output into classification results 65.

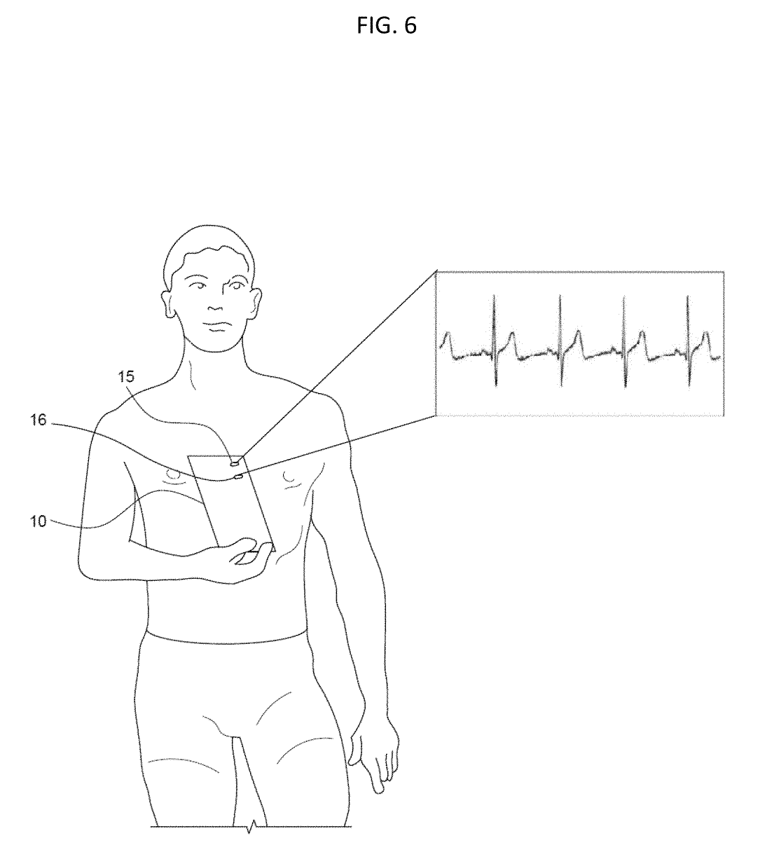

In reference to FIG. 6, an embodiment is shown wherein Non-Contact Bio Potential (NCBP) sensor data is used for quick arrhythmia detection in a user or patient.

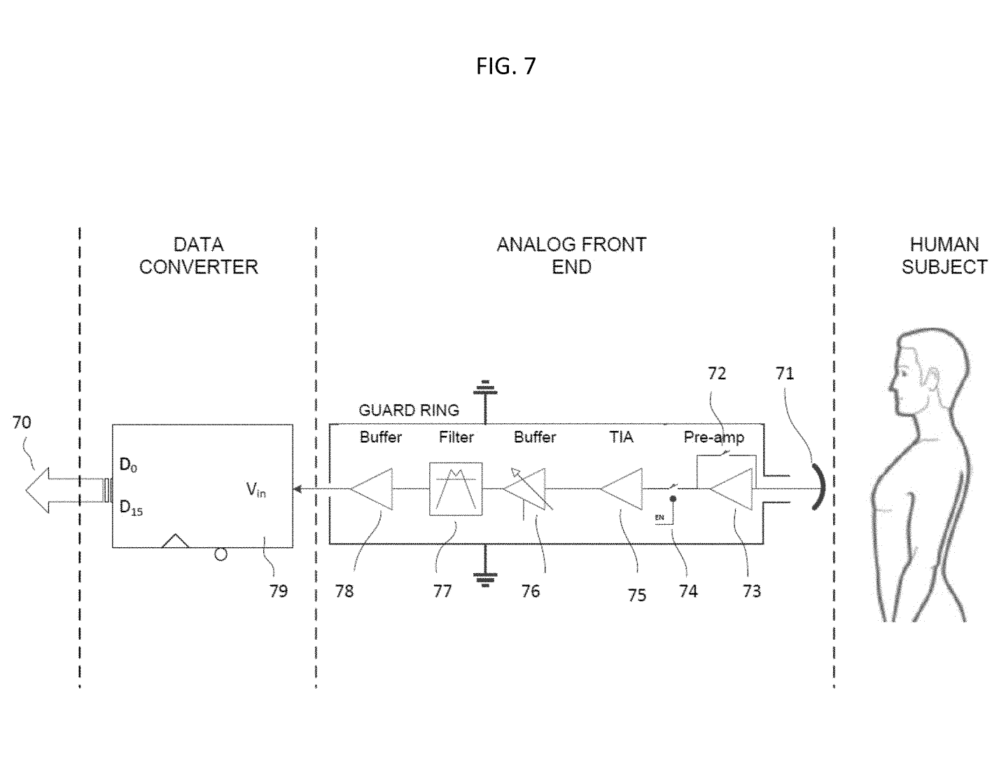

In reference to FIG. 7, a schematic of the NCBP sensor construction is represented, according to an embodiment of the present invention. In the embodiment, an electrode 71 is provided to capture electrical signals from the heart. The electric signals captured by the electrode 71 are then sent to the pre-amplifier 73 which is provided with bypass switch 72. After amplification, the signal is passed to the transimpedance amplifier 75 when the NCBP enablement switch 74 is in an on position. From the transimpedance amplifier 75, the electric heart signal is sent to a programmable gain buffer 76 before being filtered by a filter circuit 77. The filtered signal is then transmitted to the output stage buffer 78 before output to a single channel, low noise, analog data converter (ADC) 79 where it is processed before reaching the sensor subsystem.

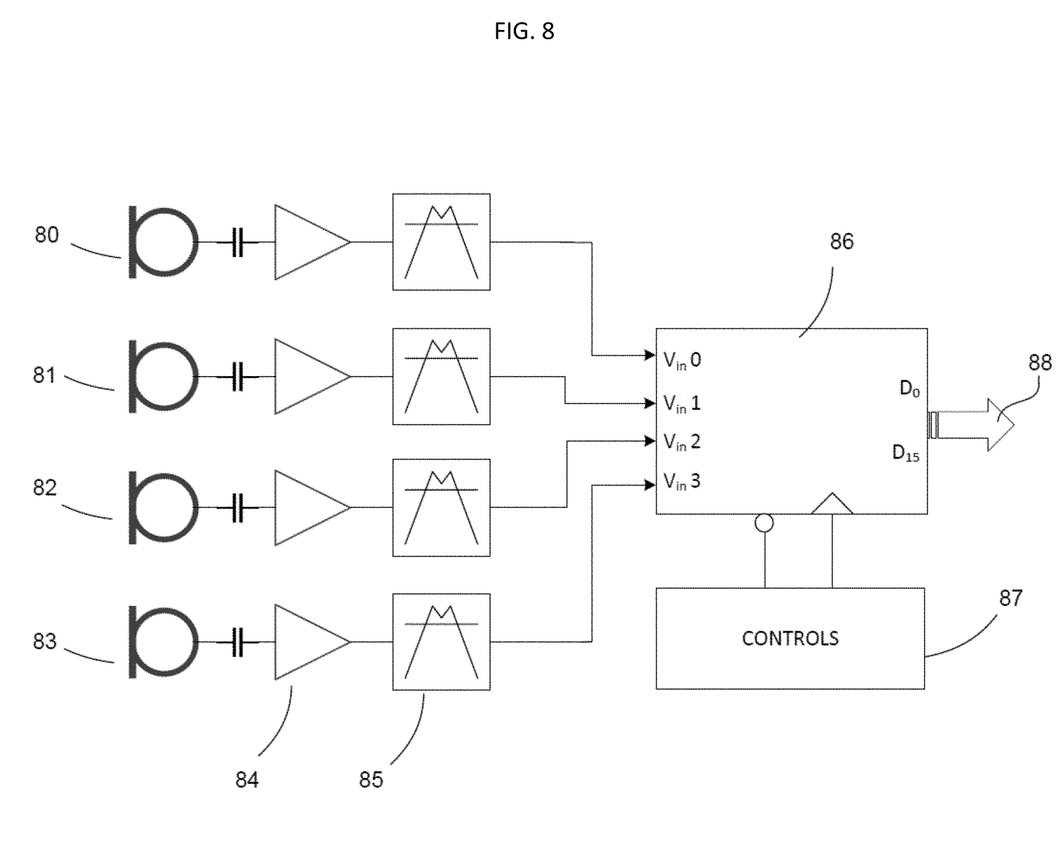

In reference to FIG. 8, a schematic diagram of the quadraphonic acoustic sensor is shown, according to an embodiment of the present invention. In an embodiment, the multi-channel phonocardiogram sensor is provided with four sensors comprised of surface pressure transducers. In an embodiment, aortic sensor 80, pulmonary sensor 81, tricuspid sensor 82, and mitral sensor 83 are provided to capture the respective heart valve signals. The signal captured by the sensors is then transmitted to a pre-amplifier 84 before transmission to a filter 85. The filtered and amplified signals are then received by an analog data converter (ADC) 86. In an exemplary embodiment, the ADC 86 is a four channel, 16-bit ADC. A control circuit 87 is in communication with the ADC 86 to enable or disable sensors, ADC, sequencing, etc. After processing by the ADC, the output signal 88 is transmitted to the sensor subsystem.

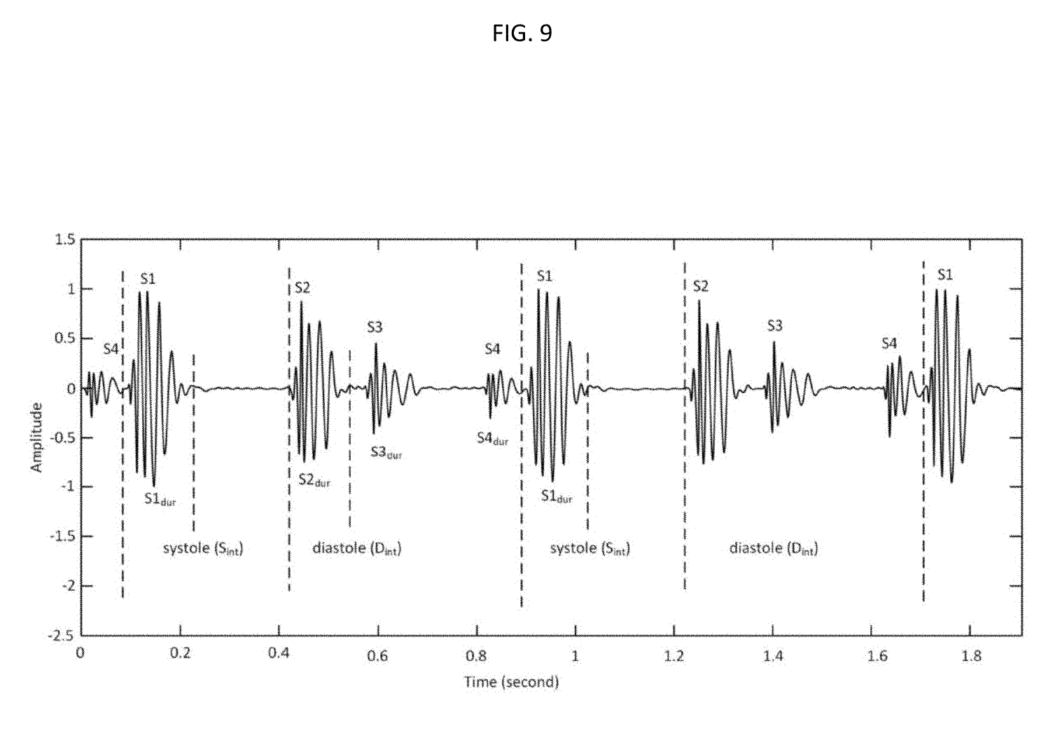

In reference to FIG. 9, an example signal output from the quadrophonic acoustic sensor is shown, according to an embodiment. In the embodiment, the data acquired from sensors are represented in their amplitude displayed over time. The device allows capture of heart sound from infrasound to ultrasound frequency range.

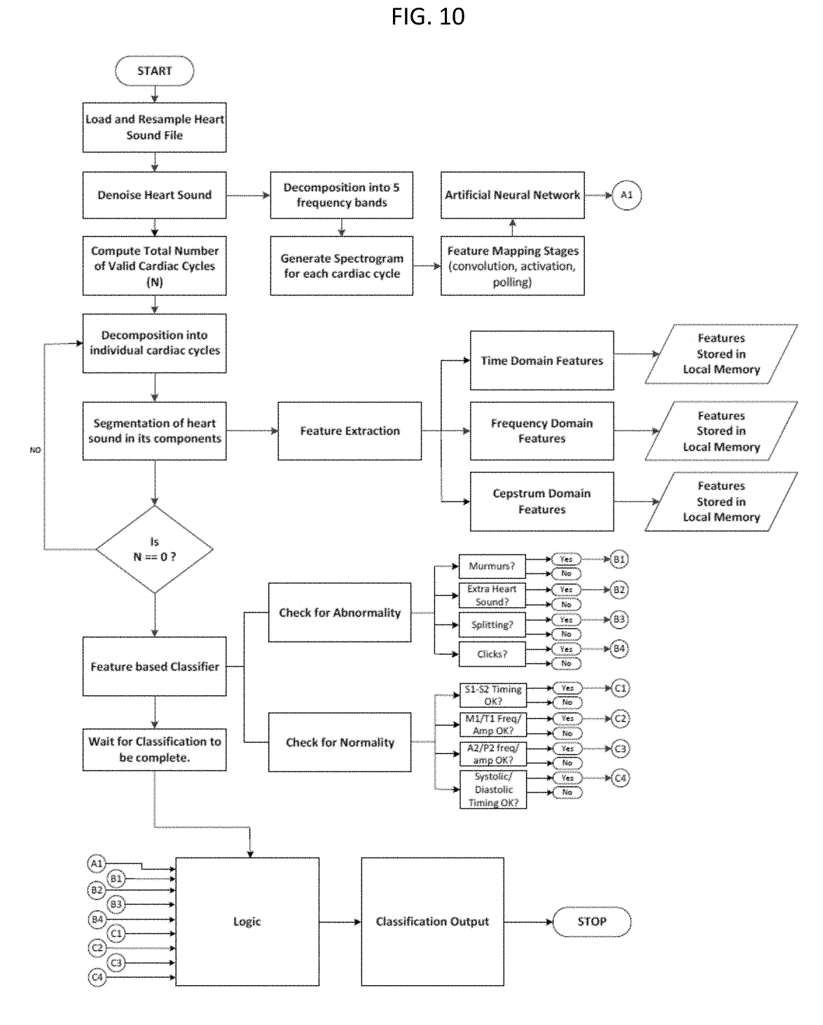

In reference to FIG. 10, a flowchart is presented to represent the processing of input signals to determine if they captured signals represent normal or abnormal behavior. In the embodiment shown, the heart sound files are loaded and then logically checked through multiple process before results are compiled in a classification output.

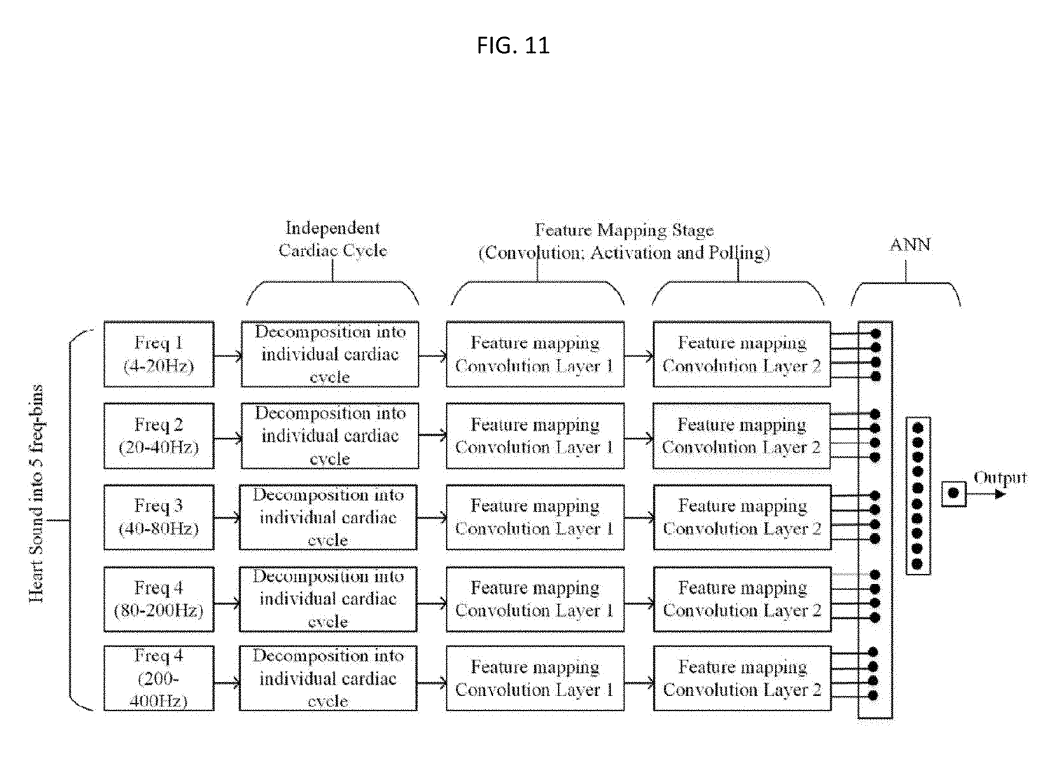

In reference to FIG. 11, a flow chart is presented to represent the processing of frequency ranges using a spectrogram and fed to a convolutional neural network (CNN) or deep neural network (DNN) to classify abnormal heart sounds. In the embodiment shown, heart sounds are separated into frequency groups. The data of each frequency group is then decomposed into individual cardiac cycles before being transmitted to the CNN for activation and polling across two convolution layers. The mapped features are then fed into the artificial neural network prior to output classification.

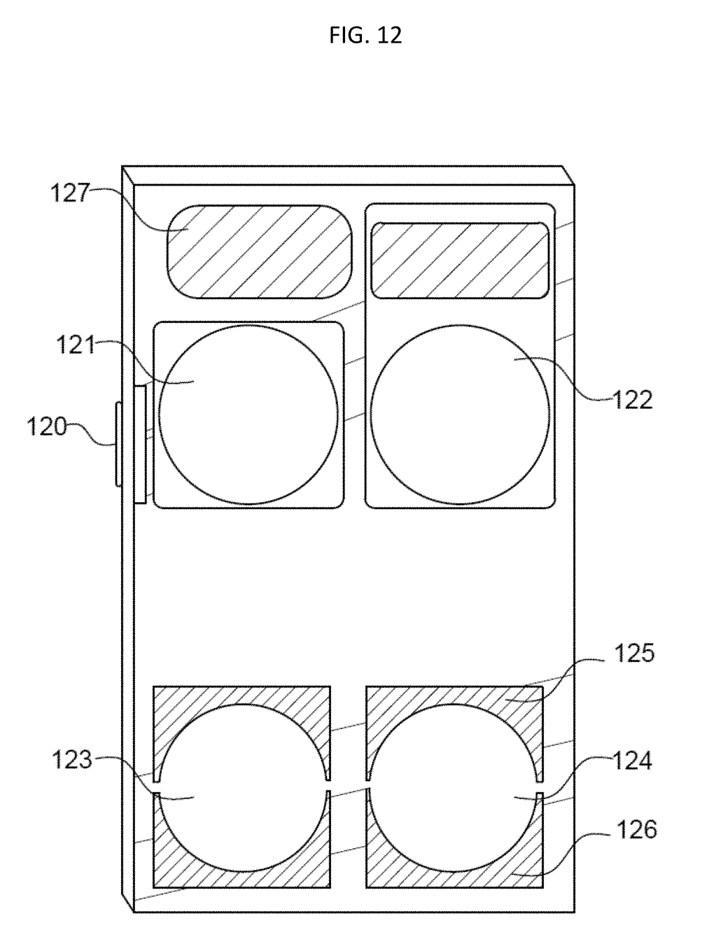

In reference to FIG. 12, an embodiment of a smart phone cover with an integrated multi-channel stethoscope system is shown. In the embodiment, the aortic sensor and pulmonary sensor are provided on an upper alignment band. According to the embodiment, the aortic sensor is further provided with a single wire ECG. The tricuspid and mitral sensors are provided on a lower alignment band, along with the ancillary sensors. In the embodiment, the multi-channel stethoscope is able to utilize the existing camera of the smart phone to assist with proper placement.

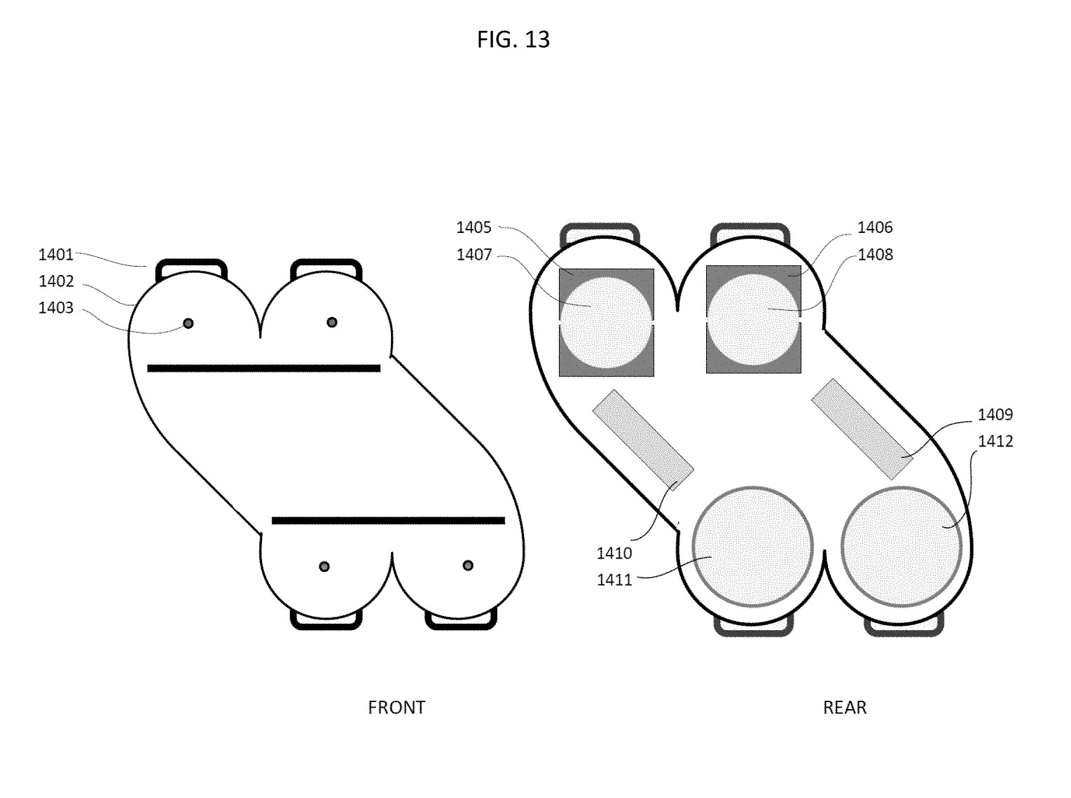

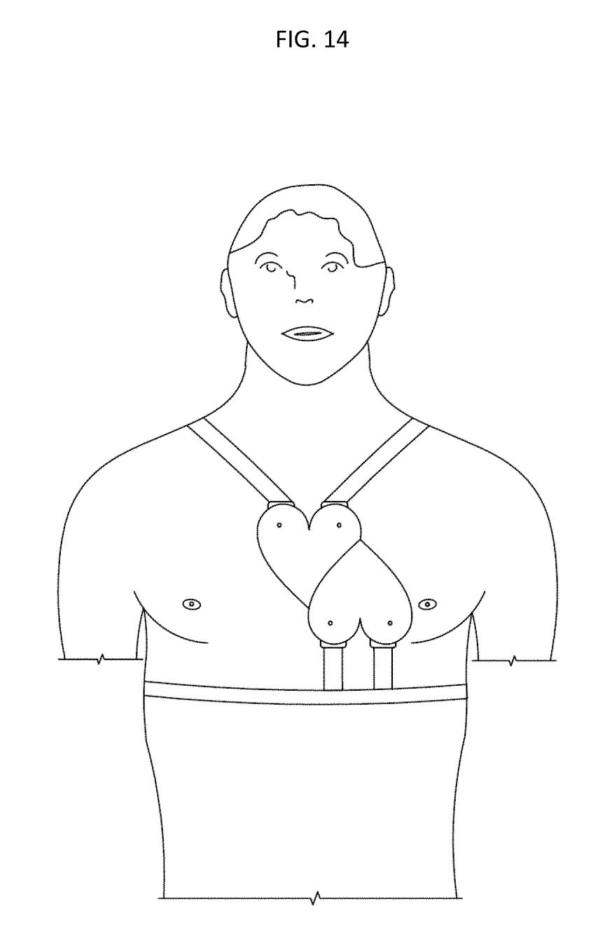

In reference to FIG. 13, an embodiment of a multi-channel stethoscope system is shown as a wearable athletic unit 1402. In the embodiment, strap hooks 1401 are provided to receive straps to secure the unit to the body of a user. The heart activity detector is further provided with activity indicator lights 1403 which indicate the sensors are actively picking up signals from the heart. In a specific embodiment the indicator lights are light emitting diodes. The rear side of the unit, to be placed against the user, is provided with aortic sensor 1408, pulmonary sensor 1407, tricuspid sensor 1412, and mitral sensor 1409 to acquire acoustic signals from the respective heart valves. In an embodiment, the pulmonary sensor 1407 is further provided with a single wire ECG lead 1405, and aortic sensor 1408 is further provided with a single wire ECG lead 1406. In an embodiment, the heart activity detector is further provided with a temperature sensor 1411 and ancillary sensors 1410. FIG. 14 shows an embodiment of the heart activity detector in use.

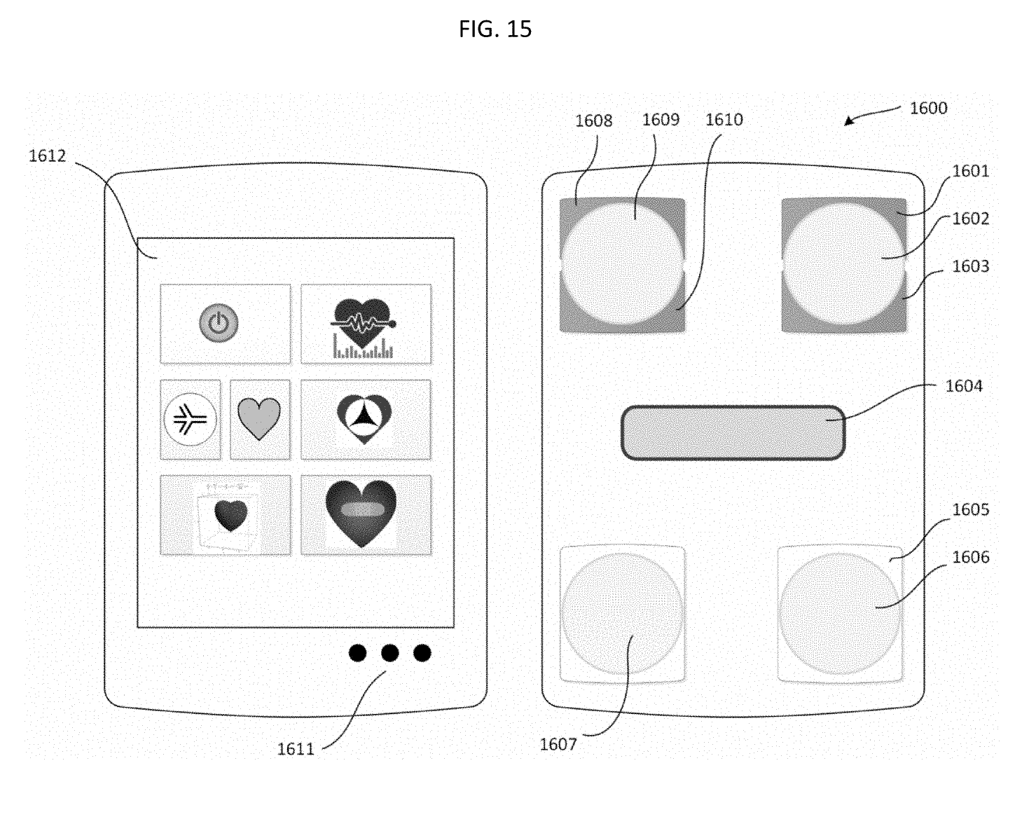

In reference to FIG. 15, an embodiment of the heart activity detector is shown as a stand-alone unit 1600. In the embodiment, the multi-channel stethoscope is provided with display 1612 and controls 1611. The rear of the unit, to be placed against a patient, is further provided with aortic sensor 1602, pulmonary sensor 1609, tricuspid sensor 1606, and mitral sensor 1607 to acquire acoustic signals from the respective heart valves. In an embodiment, the pulmonary sensor 1609 is further provided with a single wire ECG lead 1610 and ground electrode 1608, and aortic sensor 1602 is further provided with a single wire ECG lead 1601 and ground electrode 1601. In an embodiment, the rear of the unit is further provided with the ancillary sensors 1604.

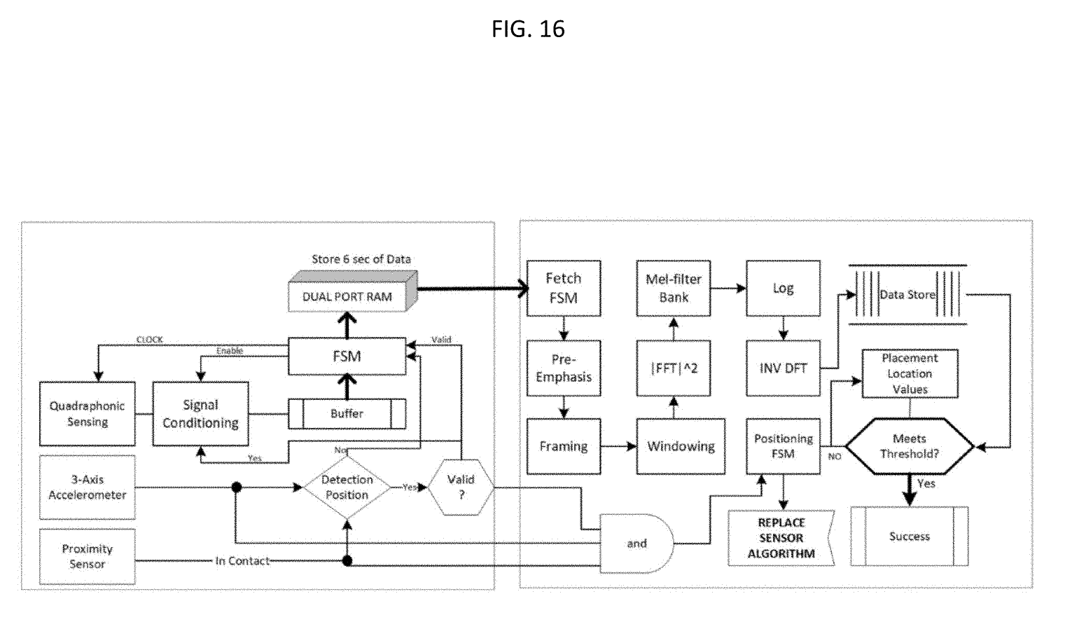

In reference to FIG. 16, a flow chart is shown representing the process of the fixed function accelerators provided in the heart activity detector, according to an embodiment of the present invention. In the embodiment, one or more phonocardiogram sensing devices is used to capture heart sound, and one or more single wire ECGs are used to capture electric signals from the heart. In an embodiment, a fixed function accelerator is used to detect captured heart signal is valid. In addition, a fixed function accelerator is used to detect if the source of heart signals is an adult, a child, or a pregnant woman. Furthermore, a fixed function accelerator is used to determine if the patient has an implanted pacemaker, defibrillator or prosthetic valve(s). Finally, a fixed function accelerator is used to determine if signal source is not human.

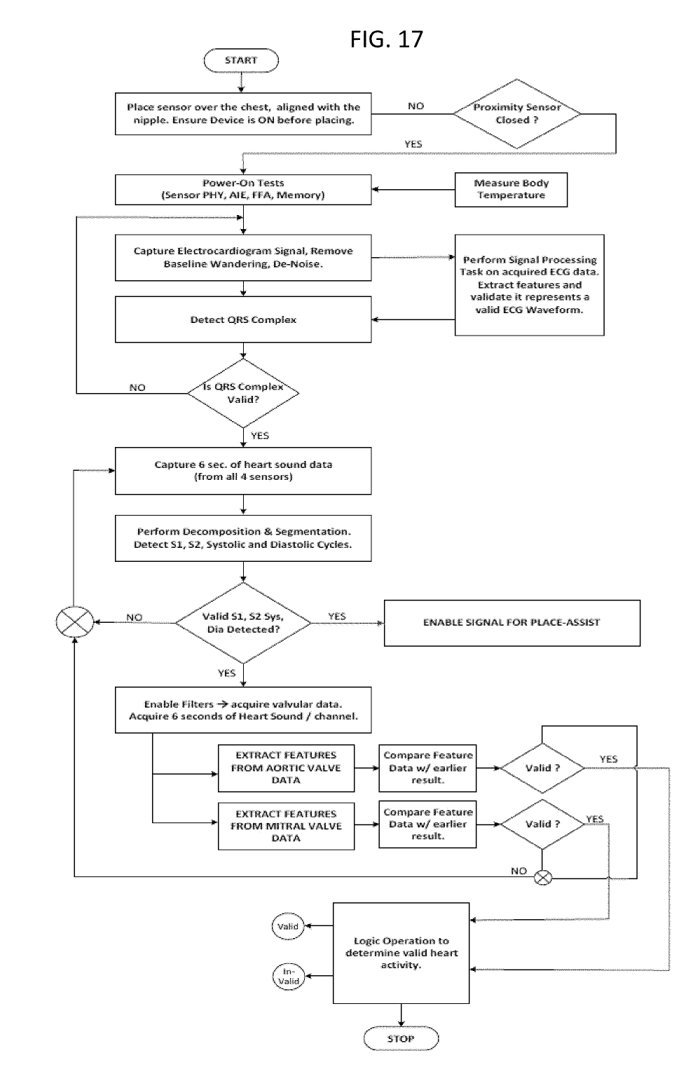

In reference to FIG. 17, a flow chart representing the execution of the overall function of the heart activity detector, according to an embodiment of the present invention. In the embodiment, the logic steps determine if the device is properly placed, based on heart sound and electrocardiogram signal recognition is valid, if all sensors are functioning, and if the heart activity recognized is valid.

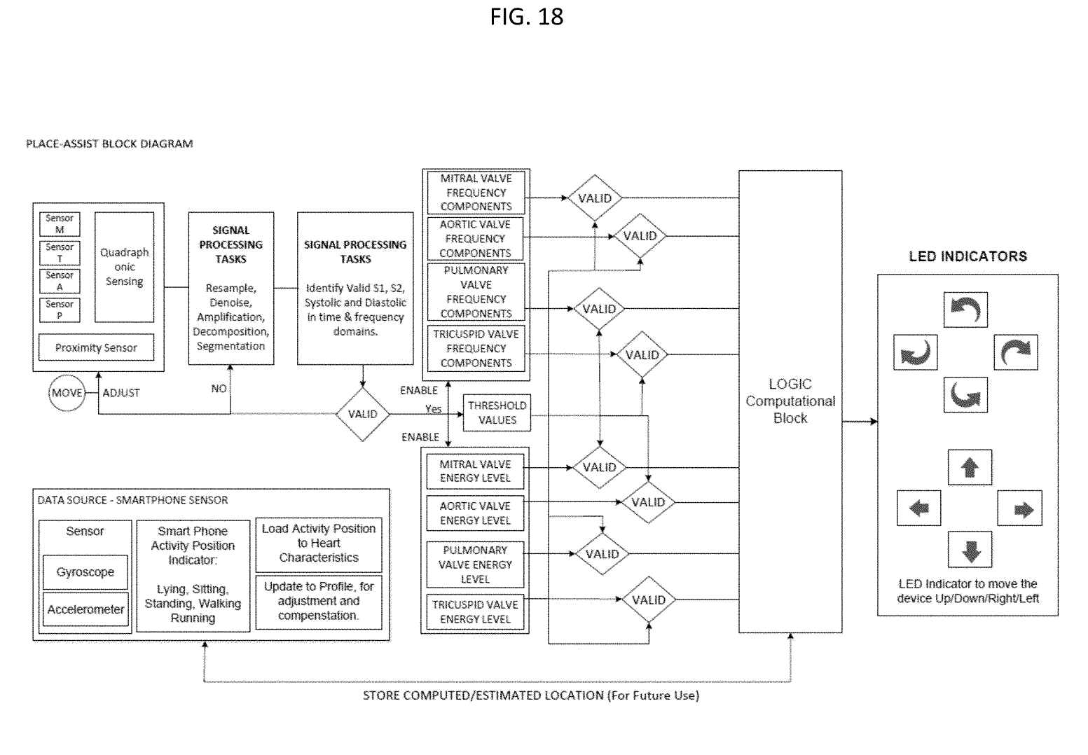

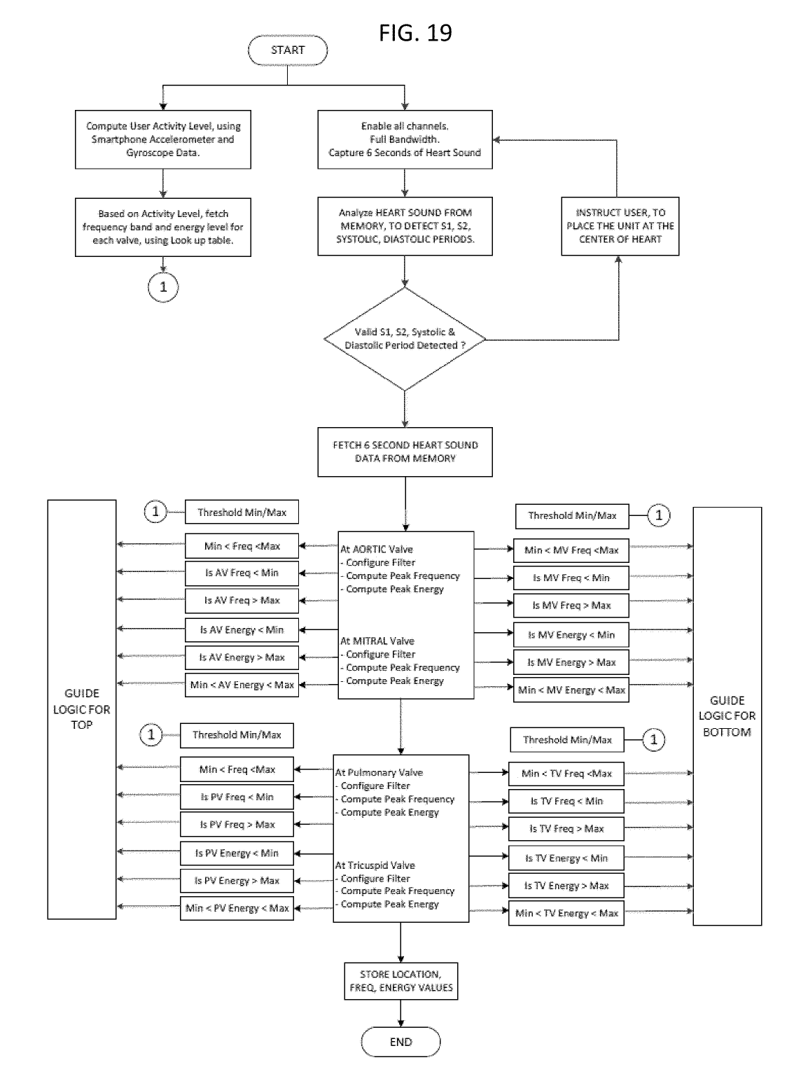

In reference to FIG. 18, a flow chart representing the place assisting mechanism of the heart activity detector is shown, according to an embodiment of the present invention. In the embodiment, input from the sensors of the heart activity detector are analyzed to determine if device is placed properly over the patient's chest. Additionally, sensors of the device, which the heart activity detector is attached or integrated to, may be utilized to ensure proper positioning. In the event that detector is out of position, the display of the device will indicate which direction the activity detector must be rotated or moved to be properly positioned. In reference to FIG. 19, a flow chart is shown to represent the algorithmic implementation of the place assisting mechanism, according to an embodiment of the present invention.

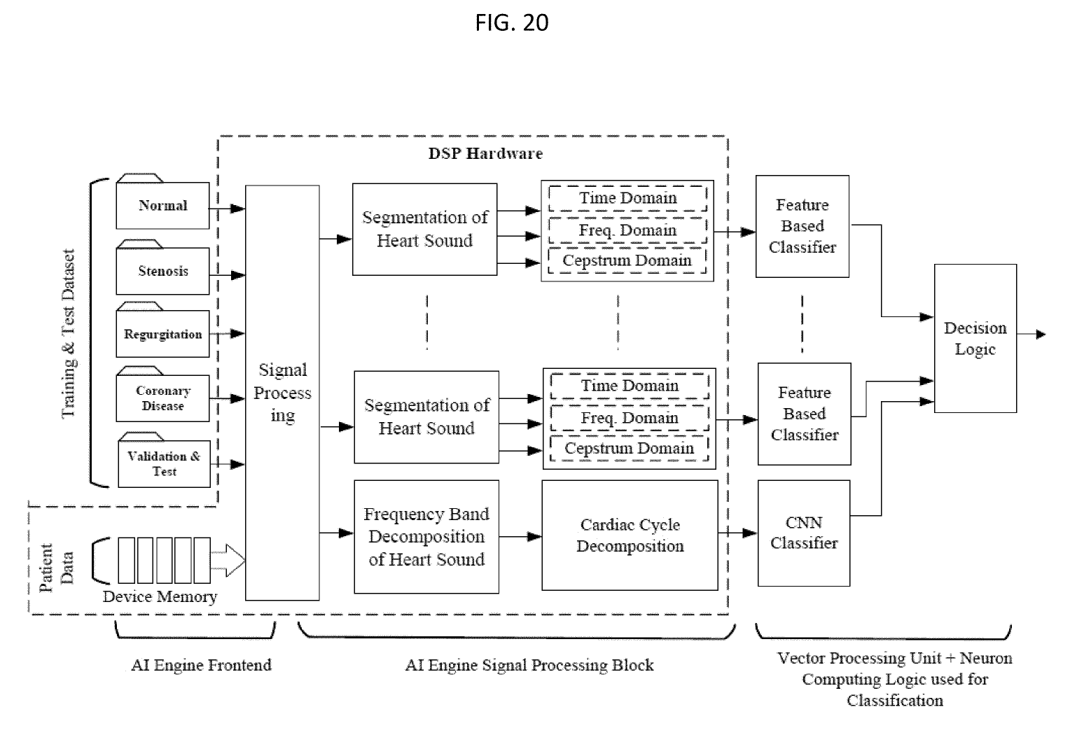

In reference to FIG. 20, a flow chart of the application of the AI engine is shown, according to an embodiment of the present invention. In the embodiment, presents classification model, which is trained using training data set, and verified using test data set. Patient's data is used to perform classification using vector processing unit and neural networks.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A heart activity detector comprising:
one or more processors;
a multi-channel stethoscope including a plurality of audio sensors, connected to the one or more processors, the plurality of audio sensors including an aortic valve sensor, a pulmonary valve sensor, a tricuspid valve sensor, and a mitral valve sensor; and
means to electronically connect the heart activity detector to a mobile communication device.

2. The heart activity detector as recited in claim 1 wherein the mobile communication device includes a smart phone, a tablet, or a computer with communication capability within a personal area network.

3. The heart activity detector of claim 1 wherein the means to electronically connect the heart activity device to the mobile communication device includes a personal area network transceiver.

4. The heart activity detector of claim 2 wherein the means to electronically connect the heart activity device to the mobile communication device includes a personal area network transceiver.

5. The heart activity detector of claim 1 further comprising one or more additional sensors consisting of a mechanical event sensor, an electrocardiogram (ECG) sensor, an ambient noise capturing microphone, a gyroscope, an accelerometer, a temperature sensor and combinations thereof.

6. The heart activity detector as recited in claim 5 wherein the ECG sensor comprises a 1-wire ECG.

7. The heart activity detector as recited in claim 1 wherein the means to electronically connect the heart activity detector to the mobile communication device includes a wired connector.

8. The heart activity detector as recited in claim 1 wherein the connector is removably connectable to the stethoscope.

9. The heart activity detector as recited in claim 1 which further includes means to connect the multi-channel stethoscope to a body.

10. A computer-readable, non-transitory, programmable product, for use in conjunction with a heart activity device comprising code for causing a processor to do the following:
cause a transceiver to transmit electronic signals from a plurality of heart sensors to a mobile communication device over a personal area network;
cause a transceiver to receive instructions and data over a personal area network; and
cause a memory to store instructions and data.

11. The computer-readable, non-transitory, programmable product as recited in claim 11 further comprising code for causing the processor to analyze data received from the plurality of heart sensors.

12. The heart activity device as recited in claim 1, wherein the heart activity detector consists of at least four or more audio sensors, which are constructed using solid state MEMS transducers, and captures simultaneous heart sound, from infrasound to ultrasound region, wherein capture is assisted by the use of mobile communication device includes a smart phone, a tablet, or a computer with communication capability.

13. The device of claim 1, wherein the heart activity detector consists of one or more pressure wave sensing devices, wherein captures muscular movement of heart at the surface of the chest and its correlation with heart sound, and wherein it measures and detects a user's activity state & convert physical activity to heart pacing rate.

14. The device of claim 1, wherein one or more above mentioned sensing devices are mounted on the back of a smartphone or smartphone cover, or as a wearable device.

15. A method for enabling heart signal capture comprises of following:
a) audio Zoom focuses on capturing and labelling heart sound components, wherein the heart sound components are associated with closing of heart valve, leaky heart valves, filling of blood and blood flow;
b) method to detect low frequency and low amplitude extra heart sound, S3 and S4;
c) method to detect if sound captured is generated by an adult human and is a valid heart signal;
d) method to place the device on human chest, to achieve optimal position and capture operation of all 4 heart valves.

16. The method of claim 15, wherein captured heart sound focused to a particular region of heart, is known to contain various components, wherein each component is decomposed and labelled to identify closing of heart valve, or leaking of heart valves, blood flow and interplay of these events.

17. The method of claim 15, wherein to detect the presence of an extra heart sound component S3 and S4, wherein Extra Heart Sounds are of Low Frequency and Low Amplitude and hard to detect with the presence of systolic and diastolic murmurs.

18. The method of claim 15, wherein a heart activity detector is used to detect if heart sound captured, is generated by a human, and wherein this method device provides inference of heart sound source as:
an adult human, a pediatric or an adult pregnant woman, a human having implanted pacemaker or defibrillator, a patient who has prosthetic valve, or a nonhuman animal,
wherein the heart activity detector is comprised of:
one or more processors;
a multi-channel stethoscope including a plurality of audio sensors, connected to the one or more processors, the plurality of audio sensors including an aortic valve sensor, a pulmonary valve sensor, a tricuspid valve sensor, and a mitral valve sensor; and
means to electronically connect the heart activity detector to a mobile communication device.

19. The method of claim 15, wherein a heart activity detector is used to enable placement of device at optimal location, or guides user to move the device until optimal location is computed, using one or more one-wire electrocardiogram sensor, one or more phonocardiogram sensor, one or more proximity sensing device and machine learning algorithms, wherein the heart activity detector is comprised of: one or more processors; a multi-channel stethoscope including a plurality of audio sensors, connected to the one or more processors, the plurality of audio sensors including an aortic valve sensor, a pulmonary valve sensor, a tricuspid valve sensor, and a mitral valve sensor; and means to electronically connect the heart activity detector to a mobile communication device.

20. The method of claim 15, wherein a heart activity detector is used to detect captured heart sound, is valid as its source is human heart, using one or more 1-wire electrocardiogram sensor, one or more phonocardiogram sensor, one or more proximity sensing device and machine learning algorithms, wherein the heart activity detector is comprised of: one or more processors; a multi-channel stethoscope including a plurality of audio sensors, connected to the one or more processors, the plurality of audio sensors including an aortic valve sensor, a pulmonary valve sensor, a tricuspid valve sensor, and a mitral valve sensor; and means to electronically connect the heart activity detector to a mobile communication device.
